(19) United States
(12) Patent Application Publication
(10) Pub. No.: US 2026/0033708 A1
(43) Pub. Date: Feb. 5, 2026

(54) DEVICES, SYSTEMS, AND METHODS FOR AUTOFLUORESENCE IMAGING

(71) Applicant: THE BOARD OF TRUSTEES OF THE LELAND STANFORD JUNIOR UNIVERSITY, Stanford, CA (US)

(72) Inventors: Stella Yang, Burlingame, CA (US); Shenglin Ye, Stanford, CA (US); Zeyi Zhou, Stanford, CA (US); Tulio Valdez, Palo Alto, CA (US)

(21) Appl. No.: 19/356,635

(22) Filed: Oct. 13, 2025

Related U.S. Application Data
(63) Continuation of application No. PCT/US24/24538, filed on Apr. 14, 2024.
(60) Provisional application No. 63/459,583, filed on Apr. 14, 2023.

(51) Int. Cl. A61B 1/06; A61B 1/00; A61B 1/04; A61B 1/227

(52) U.S. Cl. CPC A61B 1/0646; A61B 1/000094; A61B 1/00096; A61B 1/042; A61B 1/043; A61B 1/0638; A61B 1/0669; A61B 1/227

(57) ABSTRACT

Devices, systems, and methods are provided for imaging cholesteatoma, e.g., using an endoscope including a light source configured to deliver visible light at one or more narrowband wavelengths distally from the endoscope selected to cause autofluorescence in target tissue, a camera coupled to the imaging element to acquire image signals of locations beyond the distal end, and one or more filters configured to remove wavelengths of light in the image signals acquired by the camera including the one or more narrowband wavelengths of visible light.

### RELATED APPLICATION DATA

The present application is a continuation of co-pending International Application No. PCT/US2024/024538, filed Apr. 14, 2024, which claims benefit of U.S. provisional application Ser. No. 63/459,583, filed Apr. 14, 2023, the entire disclosures of which are expressly incorporated by reference herein.

### STATEMENT REGARDING FEDERALLY SPONSORED RESEARCH AND DEVELOPMENT

None.

### TECHNICAL FIELD

The present application relates to medical devices and, more particularly to devices, systems, and methods for imaging during surgical or other medical procedures, e.g., using narrowband light to excite and cause fluorescence in targeted tissue, e.g., to identify cholesteatoma within a patient's ear.

### BACKGROUND

Cholesteatoma is a proliferative lesion of keratinized epithelium overlying a fibrous stroma that occurs in the middle ear and mastoid cavity. The term “cholesteatoma” was coined in 1838 by Muller based on his belief that the lesion was composed mostly of cholesterine. Despite the term cholesteatoma being a misnomer (as this benign lesion contains neither cholesterine nor fat), it is still the most commonly used term to refer to these lesions. It is widely recognized that cholesteatomas are divided into two categories congenital and acquired. Cholesteatomas are considered congenital when they present in the absence of prior surgery or perforation and usually occur in the pediatric population. These lesions are often asymptomatic and are detected either during routine pediatric visits or when the disease is grossly advanced. When cholesteatoma cases become symptomatic, foul smelling otorrhea, otalgia, and hearing loss are common manifestations of this disease process.

Ominously, cholesteatomas if untreated can lead to severe complications such as brain abscesses, meningitis, facial nerve paralysis, and hearing loss. There is no medical treatment for cholesteatoma and it requires complete surgical removal of the lesion via tympanomastoidectomy to prevent recidivism. Unfortunately, cholesteatomas recur with rates ranging from 5 to 50% thereby necessitating multiple surgical procedures. One of the principal determinants of this high rate of recidivism is residual cholesteatoma left behind after the surgery—a direct consequence of the inability to accurately visualize the lesion margins. Thus, there is an unmet need for an imaging system to augment the real-time diagnostic information available to the surgeon in order to enable more complete resection of the lesion. Additionally, such an imaging system must allow inspection of all quadrants of the tympanic membrane and middle ear as well as provide the necessary specificity to distinguish between other lesions similar in color behind the tympanic membrane from those within, prominently myringosclerosis. The capability of performing an accurate assessment in congenital cholesteatomas is further compromised by difficulties in examining the middle ear in pediatric patients. Potential pitfalls include the presence of narrow external auditory canals and an uncooperative child.

Despite these critical challenges, the otoscope, which is the most common instrument for middle ear examination, has undergone few changes since the mid-nineteenth century. Otoscopic examination still relies on light reflecting from the tympanic membrane and interpretation of the findings by the physician. Taken together, these visualization hurdles make the diagnosis of a congenital cholesteatoma difficult for both pediatricians and otolaryngologists. Similar hurdles are faced on the operating room where white light and magnification using a microscope or endoscopes have not resulted in decreased cholesteatoma recurrence. In this context, the ability to obtain biochemical information of the tympanic membrane and middle ear lesions would provide a much-needed dimension to the diagnosis of congenital cholesteatoma.

Accordingly, devices and methods that facilitate imaging during medical procedures would be useful.

### SUMMARY

The present application is directed to medical devices and, more particularly to devices, systems, and methods for imaging during surgical or other medical procedures, e.g., using narrowband light to excite and cause fluorescence in targeted tissue, e.g., to identify cholesteatoma within a patient's ear.

Cholesteatoma is an expansile destructive lesion of the middle ear and mastoid, which can result in significant complications by eroding adjacent bony structures. The inability to accurately distinguish cholesteatoma tissue margins from middle ear mucosa tissue is the primary cause for a high rate of recidivism after surgical removal. The devices, systems, and methods described herein may facilitate distinguishing cholesteatoma tissue from mucosa tissue, e.g., to ensure that the cholesteatoma tissue is identified and removed entirely during a surgical procedure and/or to confirm that no new cholesteatoma tissue has developed or remains after a previous surgery.

In accordance with one example, an imaging device is provided that includes an elongate member comprising a proximal end, a distal end sized for introduction into the body, and an imaging element carried by the distal end; a light source coupled to the imaging device to deliver visible light at one or more narrowband wavelengths distally from the distal end selected to cause autofluorescence in target tissue; a camera coupled to the imaging element to acquire image signals of locations beyond the distal end; and one or more filters configured to remove wavelengths of light in the image signals acquired by the camera including the one or more narrowband wavelengths of visible light.

In accordance with another example, a method is provided for imaging within a passage of a subject's body that includes introducing an imaging device into a target location within the body; delivering visible light at one or more narrowband wavelengths distally from the distal end into the passage to cause autofluorescence in target tissue; and acquiring images via the imaging device within the passage to identify the target tissue.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1A is a schematic of an example an imaging device and system including an endoscope, a light source, and a camera, e.g., configured to deliver narrowband visible light to excite and cause fluorescence in targeted tissue, and acquire images of the tissue.

FIG. 1B is a side view of the imaging device of FIG. 1A.

FIG. 2 shows an exemplary experimental set-up for comparing the autofluorescence of cholesteatoma tissue to that of mucosa tissue.

FIG. 3A shows examples of narrowband illumination that may be provided by the light source of the imaging device of FIGS. 1A and 1B, e.g., including narrow bands of visible light with peak energy at 405, 450 and 520 nm.

FIG. 3B shows examples of spectral transmissivity of filters that may be included in the imaging device of FIGS. 1A and 1B, e.g., including 425, 475, and 550 nm longpass (LP) filters.

FIG. 4 shows examples of tissue fluorescence of cholesteatoma tissue compared to mucosa tissue.

FIGS. 5A-5C show cholesteatoma tissue fluorescence measurements acquired when illuminated with narrowband light, i.e., 405, 450, and 520 nm, respectively, and filtered, e.g., using one or more longpass and/or bandpass filters.

FIGS. 6A-6F are exemplary reflectance and fluorescence images of cholesteatoma and mucosa tissue samples.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

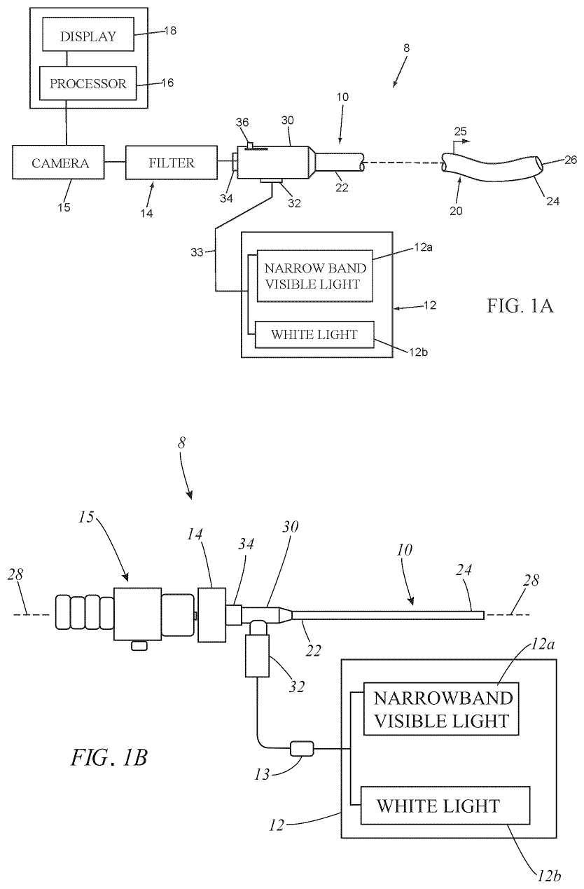

Turning to the drawings, FIGS. 1A and 1B show an example of a system 8 for imaging during a medical procedure, e.g., a surgical procedure in which one or more devices are introduced into one or more body lumens, passages, and/or regions within a patient's body. As shown, the system 8 includes an imaging device 10, e.g., an endoscope or boroscope including a light source 12, one or more filters 14, and a camera 15. Optionally, the system 8 may include one or more additional components, e.g., a processor 16 coupled to the camera 15 for processing images from the camera 15 and/or a display 18 coupled to the processor 16 for presenting images to the user, as described further elsewhere herein. Although the system 8 is shown including an endoscope 10, it will be appreciated that other imaging devices capable of being introduced into a patient's body may be provided including the light source 12, camera, 15, and/or other components of the examples described herein.

Generally, the endoscope 10 includes a shaft or elongate member 20 including a proximal end 22 including a handle or hub 30, a distal end 24 sized for introduction into a patient's body, and an imaging element 26 carried by the distal end 24. The elongate member 20 may include one or more lumens (not shown) extending between the proximal and distal ends 22, 24. For example, a fiber lumen may be provided within which one or more optical fibers, e.g., a multiple fiber bundle or individual fibers, may be received that are coupled to the imaging element 26 for delivering light and/or acquiring images beyond the distal end 24, as described further elsewhere herein. Alternatively, a camera chip, e.g., a CCD, CMOS, and the like, may be provided on the distal end 24, one or more leads (not shown) may extend proximally through the elongate member 20, which may be coupled to the processor 16 to deliver image signals from the camera, as described further elsewhere herein.

Optionally, an infusion and/or instrument lumen may be provided that extends from a port on the hub 30 to an outlet in the distal end 24 (not shown), e.g., to deliver fluids beyond the distal end 24 and/or introducing one or more instruments through the endoscope 10 during a procedure.

The elongate member 20 may be substantially flexible, semi-rigid, and/or rigid along its length, and may be formed from a variety of materials, including plastic, metal, and/or composite materials, as is well known to those skilled in the art. For example, the elongate member 20 may be substantially rigid along its entire length, e.g., to facilitate introducing the distal end 24 into a patient's ear canal, while allowing manipulation using the handle or hub 30.

Alternatively, the elongate member 20 may be substantially flexible along a distal portion 25 terminating at the distal end 24 to facilitate advancement through tortuous anatomy, and/or may be semi-rigid or rigid adjacent the proximal end 12 to enhance pushability and/or torqueability of the endoscope 10 without substantial risk of buckling or kinking. Optionally, the endoscope 10 may include one or more wires or other steering elements slidably received within respective steering lumen(s) (not shown) extending from the proximal end 22 to a fixed location within or at the distal end 24 to allow the distal portion 25 to be bent or otherwise steered, e.g., to allow the distal end 14 to be introduced into a patient's body. In this example, an actuator, e.g., a slider or rotating dial (not shown), may be provided on the hub 30 that is coupled to the steering element(s) to manipulate the shape and/or curvature of the distal end 14 during introduction. Alternatively, the distal portion 25 may be sufficiently flexible that the distal portion 25 may be advanced over a guidewire or other rail previously introduced into the patient's body, e.g., via an instrument lumen of the endoscope 10 (not shown). In a further alternative, at least the distal end 14 may be malleable to allow a shape of the distal end 14 to be adjusted and set before introduction into the patient's body.

The hub 30 may also include one or more connectors, e.g., for coupling the light source 12, filter(s) 14, and/or camera 15 to the endoscope 10. For example, as shown in FIG. 1B, a first connector 32 may be provided on the hub 30 configured to connect to a fiberoptic cable 33 to couple an external light source 12 to the optical fiber(s) within the elongate member 20, thereby optically coupling the light source 12 with the imaging element 26. In addition, a second connector 34, e.g., a C-mount and the like, may be provided on the hub 30 configured to optically couple the filter 14 and/or camera 15 to the hub 30.

In one example, the imaging element 26 may include one or more lenses, filters, and the like to facilitate transmission of light from the light source 12 beyond the distal end 24 and/or acquire images by the camera 15, e.g., to provide a desired field of view beyond the distal end 24. The imaging element 26 may be optically coupled to one or more optical fibers (not shown) extending between the distal and proximal ends 24, 22.

One or more optical couplings (not shown) may also be provided within the hub 30, e.g., to direct light from the light source 12 distally through the fiber(s) to the imaging element 26 and/or to direct light from the imaging element 26 to the camera 14 to acquire optical image signals beyond the distal end 24. Alternatively, separate fibers may be provided that extend from the first connector 33 to the distal end 24 and from the second connector 34 to the distal end 24 to transmit light and receive image signals in the separate fibers. In another alternative, a camera, e.g., a CMOS, CCD, and the like, may be carried on the distal end 24 and one or more wires or cables (not shown) may extend proximally from the camera to the hub 30 that may be connected to the processor 16 and/or display 18 via an electrical connector on the hub 30, e.g., for coupling the device 10 to a power source, such as a battery or controller (not shown). Optionally, one or more filters, lenses, and the like (not shown) may be optically coupled to the camera.

Optionally, one or more cables (not shown) may be coupled to the hub 30, which may be connected to an external power source, e.g., a battery, an A/C wall source, and the like to provide electrical power to components of the device 10. Alternatively, a battery or other power source may be carried within the hub 30 to provide power to the components.

With continued reference to FIG. 1A, the light source 12 may include one or more LEDs, laser, or other light sources configured to deliver one or more desired wavelengths of light via the device 10, e.g., from the imaging element 26 on the distal end 24. Alternatively, one or more of the light sources may be carried on the distal end 24 and one or more wires or cables may extend proximally from the distal end, e.g., to a power source (not shown) connected to the hub 30, to deliver power and/or otherwise activate/deactivate the light source(s).

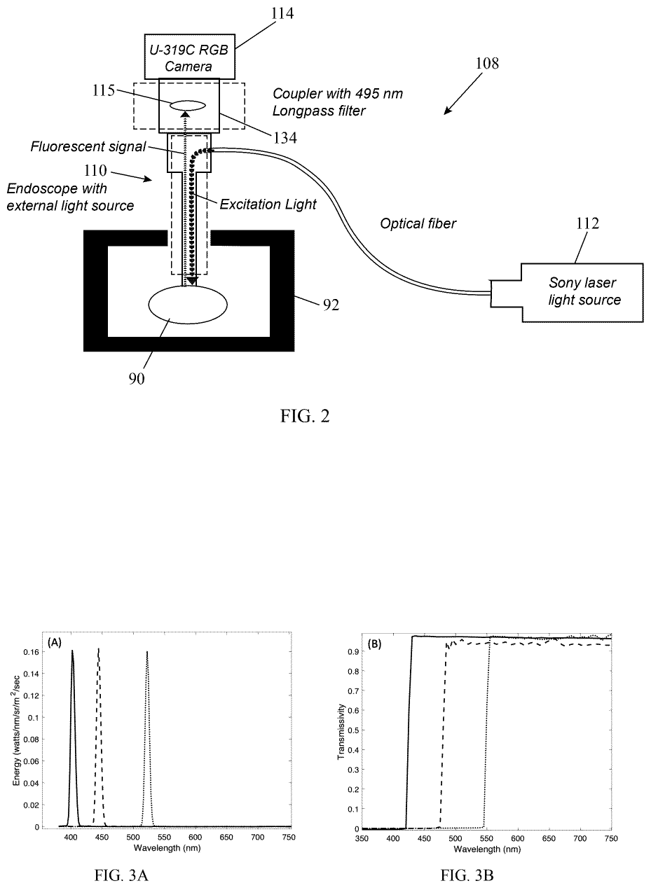

For example, the light source 12 may include one or more lasers configured to deliver one or more narrowband wavelengths of visible light, e.g., at peak wavelengths of about 405, 450, and 520 nanometers, e.g., as shown in FIG. 2A. In particular, the light source 12 may be configured to deliver a narrowband wavelength of visible light at wavelengths for example, at a peak wavelength of about 405 or 450 nanometers, selected to cause target tissue, e.g., tissues containing keratin such as cholesteatoma tissue, to fluoresce. Alternatively, the light source may be configured to transmit a broad wavelength of visible light and one or more filters (not shown) may be optically coupled between the light source and the imaging element 26 such that only the narrowband wavelength(s) is (are) transmitted from the distal end 24 of the endoscope 10.

In addition or alternatively, the endoscope may have different angles to illuminate tissue. For example, the distal end 24 may be configured such that the light transmitted by the imaging element 26 extends along an axis parallel with or offset from a longitudinal axis 28 of the endoscope 10. For example, the imaging element 26 may include one or more lenses or other features (not shown) configured to direct incident light transmitted from the distal end 24 at an angle offset from the longitudinal axis 28, e.g., by about thirty degrees) (30°, about forty five degrees) (45°, or about seventy degrees) (70°. Such offset may provide sufficient narrowband visible light to tissue beyond the distal end 24 to cause fluorescence but avoid the narrowband light reflecting back into the imaging element 26, e.g., to prevent overexposure or saturation of the camera 15.

The filter(s) 14 may be configured to remove wavelengths of light in the image signals acquired by the camera 15 including the one or more narrowband wavelengths of visible light, e.g., to prevent reflected light within the narrowband wavelengths saturating or otherwise obscuring the images. For example, the filter(s) 14 may include one or more longpass (LP) filters, bandpass (BP) filters, and/or rejection filters configured to block reflected light from the light source being received by the imaging element 26. In one example, a longpass filter, e.g., a 425 nm longpass filter, a 475 nm longpass filter, and/or a 550 nm longpass filter, may be coupled between the hub 30 and the camera 15 to block substantially all light below the designated wavelength and transmit light above the designated wavelength.

For example, if the light source 12 is configured to transmit a narrowband of light centered at 405 nanometers, a 425 nm longpass filter may be provided, while for a narrowband of light centered at 450 nm, a 475 nm longpass filter may be provided to ensure that incident light from the light source 12 transmitted by the imaging element 26 is reflected and received at the camera 15. Such combinations of narrowband incident light and filtered light received by the camera 15 may ensure that the camera 15 receives fluoresced light generated by the target tissue without being saturated by the incident light transmitted by the imaging element 26.

Alternatively, the system 8 may include other filters to filter reflected light at the incident wavelengths and allow fluoresced light to be received at the camera 15. For example, the filter(s) 14 may include one or more bandpass filters selected to transmit light within the wavelengths of the fluoresced light and rejects wavelengths outside the designated band. For example, a bandpass filter may be provided having a band centered on about 425 nanometers or 475 nanometers, e.g., having a bandwidth of about +/−ten nanometers (10 nm) or other desired bandwidth. Alternatively, a bandpass filter centered at the peak wavelength of the fluoresced light, e.g., at about 460 nanometers, may be provided if the incident light is outside the bandwidth of the filter, e.g., at a narrowband centered at 405 nanometers. In a further alternative, one or more rejection or band-stop filters may be provided that are configured to reject light at a bandwidth including the incident wavelength(s) and transmit light outside the bandwidth to allow fluoresced light to be received by the camera 15. Consequently, the one or more filters may pass fluoresced light from the tissue to the camera 15 while rejecting reflected light in the narrowband wavelength(s), e.g., to prevent overexposure or saturation of the camera 14.

Optionally, the light source 12 may also include an LED or other white light source 12b configured to deliver visible light (white or other broadband visible light for illumination) from the imaging element 26, e.g., to allow for surgical navigation when manipulating the endoscope 10 within the subject's body. Optionally, the light source 12 may also include one or more near IR LEDs or laser sources (not shown) configured to deliver near IR light, e.g., between about 600-825 nm or between about 785-808 nm, to allow excitation of ICG or other fluorescent dye administered to the subject, e.g., as disclosed in U.S. Publication No. 2022/0087592, the entire disclosure of which is expressly incorporated by reference herein.

The hub 30 may include one or more actuators, e.g., a switch 36, that may be actuated to turn desired light sources off and on. For example, a single switch 36 may be provided on the hub 30 that may be moved between a first/off position where the light source 12 is completely off or isolated from the fiber(s) within the elongate member 20, a second position where the narrowband light source 12a is activated (without activating the visible light source 12b), and a third position where the visible light source 12b is activated (with the narrowband light source 12a remaining on or turning off). For example, the switch 36 may be used to alternate between activating the narrowband light source and the visible light source. Alternatively, separate switches or other actuators 13 may be provided to selectively activate the light sources, e.g., on the hub 30, on a floor step-on switch (not shown) coupled to the hub 30, on the light source 12 itself, and the like.

The camera 15 may include a detector, e.g., a CMOS, CCD, InGaAs, or other sensor, configured to be coupled to the hub 30, e.g., via the filter 14 and a second connector 34, e.g., a C-mount, to acquire images from the imaging element 26 on the distal end 24 of the endoscope 10.

The camera 15 may be connected to a processor 16 and/or display 18, e.g., within a control box, such that signals from the camera 15 may be processed by the processor 16 for presentation on the display 18. Optionally, if desired, the light source 12 may also be provided within the control box to provide a single component to which the endoscope 10 may be connected to use the system 8. Alternatively, the camera 15 may also be provided within the control box. For example, in this alternative, a single connector may be provided on the hub 30 of the endoscope 10 that be connected to a corresponding connector on the control box to allow light (e.g., narrowband visible light, broadband visible light, and the like) to be delivered to the imaging element 26 from the light source(s) within the control box and to deliver image signals to be conveyed from the imaging element 26 (e.g., reflected and/or fluoresced light) to the camera (e.g., through the filter) within the control box (or electrical signals from a camera carried on the distal end 24 to the processor).

The system 8 may be used during a medical procedure, e.g., during a surgical procedure in which the endoscope 10 is introduced into a patient's body, e.g., into the patient's ear canal to identify cholesteatoma. Alternatively, the system 8 may be used to acquire images within other body lumens or passages within a patient's body. For example, initially, the broadband visible (e.g., white) light source 12b may be activated to provide illumination and images may be acquired to facilitate manipulation of the distal end 24 into the target location. Thus, images presented on display 18 may facilitate introduction of the distal end 24, e.g., manipulating a rigid distal end 24 into an ear or other body lumen, using one or more steering elements in the endoscope 10 to navigate the distal end 24 into a passage within the patient's body, and/or advancing the distal end 24 over a guidewire (not shown) to the target location.

For example, the distal end 24 may be introduced into a patient's ear and, once within the inner ear, the narrowband light source(s) 12a may be activated to cause autofluorescence of cholesteatoma tissue (with the broadband visible light source 12b off or remaining on), e.g., to facilitate identifying the cholesteatoma tissue compared to mucosa or other tissues. For example, the switch 36 may be actuated to activate the narrowband light source 12a and deactivate the broadband light source 12b, and images acquired by the camera 15 may be presented on the display 18, which may show cholesteatoma tissue based on the fluoresced light that is captured, while mucosa or other tissue remains dark. For example, the filter 14 may filter desired wavelengths (e.g., wavelengths below the threshold of a longpass filter or wavelengths within the bandwidth of a bandpass filter) to ensure that any fluoresced light transmitted by tissue in response to the transmitted light is received by the camera 15. Images from the camera 15 may be presented on the display 18, e.g., in real time, to allow the surgeon or other user to identify cholesteatoma tissue within the target location. If desired, the user may alternate between the visible light and the narrowband light to identify anatomical markers or otherwise identify the location of the cholesteatoma tissue.

Optionally, the processor 16 may process the images, e.g., to identify the cholesteatoma tissue in a manner that may be superimposed on white light images. For example, the processor 16 may alternately activate the narrowband and visible light sources 12a, 12b to acquire images along a common field of view and then combine the images to facilitate identifying the cholesteatoma tissue. Alternatively, the user can manually activate either light source and observe the acquired images on the display 18.

In a further alternative, the system 8 may include an exoscope configured to generate three-dimensional images based on the image signals received by the camera 15. The processor 16 may process signals from the camera to generate three-dimensional images to allow a surgeon or other operator to perform a procedure based on the presented images.

The images presented on the display 18 may be used during a surgical procedure, e.g., to allow visualization while introducing a surgical instrument (not shown) into the patient's body, e.g., ear canal, to remove the cholesteatoma. For example, one or more scalpels, forceps, cautery devices and the like may be introduced, e.g., through a lumen of the endoscope 10 and/or separately, to perform a mastoidectomy to remove the cholesteatoma tissue. The images may be used during the procedure to identify the cholesteatoma tissue and/or to confirm the entire cholesteatoma has been removed. Once the procedure is completed, e.g., after confirming all cholesteatoma tissue has been removed, the endoscope 10 may be removed.

### Example

Turning to FIG. 2, an experimental set-up 108 is shown that was used to compare the autofluorescence of cholesteatoma tissue compared to mucosa tissue. Tissue samples 90 were secured in a black enclosure 92 to avoid any ambient light reflection. An endoscope 110 equipped with an external light source 112, e.g., a Sony laser diode video scope illumination unit (transmitting narrowband visible light, represented by thick arrows pointing down) was applied to excite and collect the fluorescence signal from the tissue samples (represented by thin arrows pointing up). A U-319C RGB camera 114 was used as the image sensor to capture the fluorescence image. An optical coupler 134 was used to connect the endoscope and camera to avoid light leakage. A 495 nm longpass filter 115 was embedded into the optical coupler and placed in front of the camera 114 to block the excitation wavelength and any other reflected light.

FIG. 5A plots the PR-670 spectroradiometric measurements that were made when several different cholesteatoma tissue samples were illuminated with a 405 nm light in combination and a 425 nm longpass filter. FIG. 5B shows the spectroradiometric measurements for the same tissues when they were illuminated with the 450 nm light and measured by the PR-670 through a 475 nm longpass filter. FIG. 5C shows the measurements made when the tissues were illuminated with the 520 light and measured with the PR-670 through a 550 nm filter.

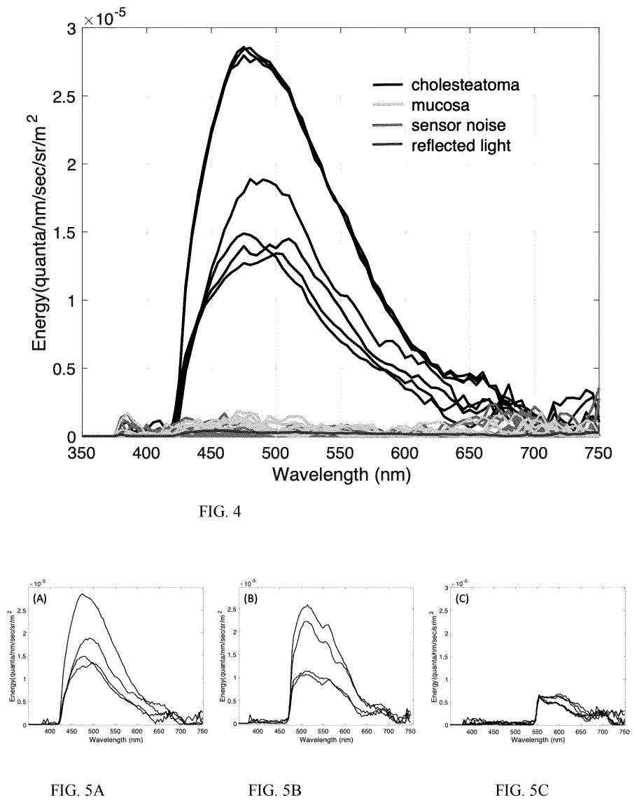

To confirm that the spectroradiometric measurements shown in FIGS. 5A-5C are the result of tissue autofluorescence and not tissue reflectance, the amount of light was estimated that would be expected to be reflected from cholesteatoma tissue. This estimate was obtained in the following way. First, the spectral energy of each light was multiplied with the filter transmittance of the longpass filter. This estimated the maximum spectral energy in each light that could be reflected from a diffusely reflecting surface. To estimate the amount of light that could be reflected from cholesteatoma tissue, the maximum reflected light was multiplied with the measured cholesteatoma tissue reflectance. (Tissue reflectance measurements are based on spectrophotometric measurements of cholesteatoma tissue illuminated by a halogen lamp divided by the spectrophotometric measurements of a white calibration surface also illuminated by the halogen and placed in the same position as the cholesteatoma tissue.) FIG. 4 shows that the measurements of cholesteatoma tissue autofluorescence for the 405 nm light is greater than the light that could be reflected from cholesteatoma tissue.

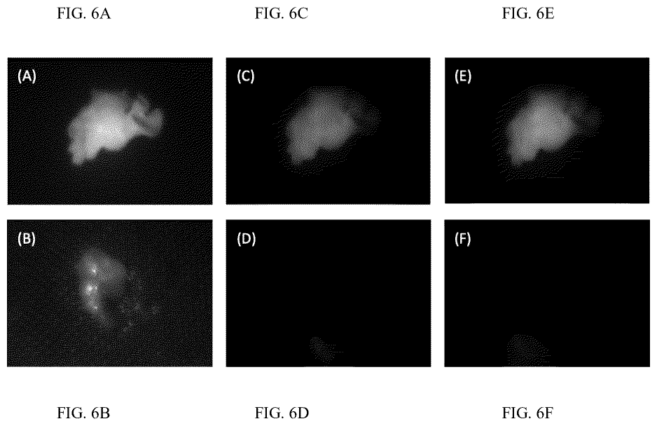

FIGS. 6A-6F show exemplary images of tissue structures imaged using the set-up of FIG. 2 using a U-319C camera to generate images of cholesteatoma and mucosa tissue reflectance and fluorescence. Reflectance images were obtained by illuminating the tissue with a white light and capturing camera images with no filter in place. Fluorescence images were obtained by illuminating the tissue with 405 nm narrowband light or with 450 narrowband light and capturing camera images with a 495 nm long pass filter in place.

To visualize tissue reflectance, the cholesteatoma (FIG. 6A) and mucosa (FIG. 6B) tissue samples were illuminated with white light and captured with no filter. Tissue fluorescence images were measured when the same cholesteatoma (FIG. 6C) and mucosa (FIG. 6D) tissue samples were illuminated with the 405 nm light and captured with a 495 nm longpass filter on the U-310c digital camera, using a 500 millisecond exposure duration. Tissue fluorescence images were also measured when the same cholesteatoma (FIG. 6E) and mucosa (FIG. 6F) tissue samples were illuminated with the 450 nm light and captured with a 495 nm longpass filter on the camera and a 500 millisecond exposure duration.

The camera images illustrate that it is possible detect fluorescence in the cholesteatoma tissue samples but not in the mucosa tissue samples. This result is consistent with the spectroradiometric measurements of cholesteatoma and mucosa tissue that are shown in FIG. 4. Since the mucosa tissue does not fluoresce under 405 or 450 nm light, the camera images of mucosa tissue should be black, as they are.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. An imaging device, comprising:
an elongate member comprising a proximal end, a distal end sized for introduction into the body, and an imaging element carried by the distal end;
a light source coupled to the imaging device to deliver visible light at one or more narrowband wavelengths distally from the distal end selected to cause autofluorescence in target tissue;
a camera coupled to the imaging element to acquire image signals of locations beyond the distal end; and
one or more filters configured to remove wavelengths of light in the image signals acquired by the camera including the one or more narrowband wavelengths of visible light.

2. The device of claim 1, wherein the light source is configured to generate narrowband visible light at a peak wavelength of about 405 nanometers.

3. The device of claim 1, wherein the light source is configured to generate narrowband visible light at a peak wavelength of about 450 nanometers.

4. The device of claim 1, wherein the light source is configured to generate narrowband visible light at both a peak wavelength of about 405 nanometers and a peak wavelength of about 450 nanometers.

5. The device of claim 1, wherein the light source comprises one or more LEDs, lasers, or halogen lights.

6. The device of claim 1, wherein the one or more filters comprise one or more longpass filters.

7. The device of claim 6, wherein the one or more filters comprise a 425 nm longpass filter.

8. The device of claim 6, wherein the one or more filters comprise a 475 nm longpass filter.

9. The device of claim 6, wherein the one or more filters comprise a 550 nm longpass filter.

10. The device of claim 1, wherein the one or more filters comprise one or more bandpass filters configured to reject light at the one or more narrowband wavelengths and transmit light at one or more wavelengths of fluoresced light of the target tissue.

11. The device of claim 10, wherein the one or more bandpass filters are configured to transmit light at a bandwidth centered at one of about 425 nm and 475 nm.

12. The device of claim 11, wherein the one or more bandpass filters has a bandwidth of about +/−ten nanometers around the bandwidth.

13. The device of claim 10, wherein the light source is configured to generate narrowband visible light at a peak wavelength of about 405 nanometers, and wherein the bandpass filter is configured to transmit light at a wavelength of about 460 nanometers and reject light at the peak wavelength.

14. The device of claim 10, wherein the light source is configured to generate narrowband visible light at a peak wavelength of about 450 nanometers, and wherein the bandpass filter is configured to transmit light at a wavelength of about 460 nanometers and reject light at the peak wavelength.

15-25. (canceled)

26. A system for imaging within a passage of a subject's ear to identify cholesteatoma tissue, comprising:
an elongate member comprising a proximal end, a distal end sized for introduction into the body, and an imaging element carried by the distal end;
a light source coupled to the imaging device to deliver visible light at one or more narrowband wavelengths distally from the distal end selected to cause autofluorescence in target tissue;
a camera coupled to the imaging element to acquire image signals of locations beyond the distal end;
one or more filters configured to remove wavelengths of light in the image signals acquired by the camera including the one or more narrowband wavelengths of visible light a processor coupled to camera to process the image signals; and
a display coupled to the processor to display images based on the image signals.

27. The system of claim 26, wherein the light source is configured to generate narrowband visible light at a peak wavelength of one or both of about 405 nanometers and 450 nanometers.

28. The system of claim 26, wherein the light source is configured to generate narrowband visible light at a peak wavelength of about 405 nanometers.

29. The system of claim 26, wherein the light source is configured to generate narrowband visible light at a peak wavelength of about 450 nanometers.

30. The system of claim 26, wherein the one or more filters comprise one or more longpass filters.

31-41. (canceled)

42. A method for imaging within a passage of a subject's body, comprising:
introducing an imaging device into a target location within the body;
delivering visible light at one or more narrowband wavelengths distally from the distal end into the passage to cause autofluorescence in target tissue; and
acquiring images via the imaging device within the passage to identify the target tissue.

43-45. (canceled)
